(19) United States
(12) Patent Application Publication
(10) Pub. No.: US 2020/0121495 A1
(43) Pub. Date: Apr. 23, 2020

(54) DEVICES AND METHODS FOR SURGICAL INDUCTION HEATING

(71) Applicant: Surgical Induction, LLC, Atlanta, GA (US)

(72) Inventors: Stephen James Nelson, New Haven, CT (US); Nick N. Patel, Atlanta, GA (US)

(21) Appl. No.: 16/657,288

(22) Filed: Oct. 18, 2019

Related U.S. Application Data
(60) Provisional application No. 62/748,913, filed on Oct. 22, 2018.

(51) Int. Cl. A61F 7/00

(52) U.S. Cl. CPC A61F 7/007; A61F 2007/009; A61F 2007/0087

(57) ABSTRACT

Systems containing an electrically conductive implant, and a device for treating the implant via induction heating are described. The device includes v. The one or more coils contain an electrically conductive material. The one or more coils can contain one or more connecting portions, and one or more heating portions. Typically, the one or more heating portions have an overall shape that conforms to one or more exposed surfaces of implants. Methods of treating an electrically conductive implant in a patient's body using the device are also described. The method includes placing the heating portions of the one or more coils in a position that is in close proximity to or in contact with one or more exposed surfaces of an implant, and activating the power supply to provide an alternating magnetic field to heat the implant.

### CROSS-REFERENCE TO RELATED APPLICATION

This application claims priority to provisional application No. 62/748,913, filed Oct. 22, 2018, the disclosure of which is incorporated herein by reference in its entirety.

### FIELD OF THE INVENTION

The invention is generally in the field of induction heating, particularly methods and devices for using induction heating to treat implants inside a patient's body.

### BACKGROUND OF THE INVENTION

Infections of implants inside a patient's body, such as periprosthetic and peri-implant infections, are difficult to treat. When such an infection occurs, bacteria produce a biofilm on the implant, making the bacteria impenetrable to antibiotics and surgical lavage.

In order to adequately treat peri-implant infections, removal of the involved hardware is often required, which often results in further complications in patients, such as bone loss, loss of fixation, and/or long operative times.

There remains a need for devices and methods for effectively preventing and treating implant infections, which allow an implant to be maintained at its position in the patient's body.

Therefore, it is an object of the present invention to provide devices for treating implants in a patient's body to prevent the development or growth of biofilm and/or to remove biofilm.

It is a further object of the present invention to provide systems for to preventing the development or growth of biofilm and/or to removing biofilm from an implant in a patient's body.

It is another object of the present invention to provide methods for treating implants in a patient's body to prevent the development or growth of biofilm and/or to remove biofilm.

It is another object of the present invention to provide methods for facilitating the removal of an implant from a patient's body.

### SUMMARY OF THE INVENTION

Systems containing an electrically conductive implant and a device for treating the implant via induction heating are described herein. Methods of treating an electrically conductive implant in a patient's body using a device are also described.

The implant typically has a shape and size suitable to be placed in the knee, the hip, the shoulder, or the elbow of a patient's body.

The device includes one or more coils. The one or more coils contain an electrically conductive material. The one or more coils include one or more connecting portions, and one or more heating portions. Typically, the one or more heating portions have an overall shape that conforms to one or more exposed surfaces of implants. Optionally, the device includes a hand unit, and/or a power supply.

The method typically includes inserting one or more heating portions into the patient's body; placing the heating portions in a first position that is in close proximity to or in contact with one or more exposed surfaces of an implant; and activating the power supply to provide an alternating magnetic field to heat the implant. Optionally, the method includes maintaining the heating portions in the first position for a sufficient period of time to achieve a predetermined temperature of the one or more exposed surfaces of the implant, and to maintain the implant at the predetermined temperature for up to about 5 minutes. This method may be used to remove biofilm from the implant and/or reduce bacterial burden of the implant.

In some embodiments, the implant is heated as described above and the heating is effective to disrupt the implant-bone, bone-cement, and/or implant-cement interface and facilitate removal of the implant from the patient. This method can be used in a revision surgery.

### BRIEF DESCRIPTION OF THE DRAWINGS

FIGS. 1A-1C depict different views of an exemplary tibial implant component and an exemplary surgical induction heating device, where the heating portions, as a group, have an overall shape similar to “T.” FIG. 1A illustrates an oblique view of the T-shaped heating portions attached to the handpiece and inserted so that the heating portions are in close proximity to the exposed surface of the implant. FIG. 1B illustrates a magnified view. FIG. 1C illustrates a side view. FIG. 1D depicts an exemplary tibial implant component.

FIG. 2 depicts a side view of an exemplary tibial implant component that is the same as depicted in FIGS. 1A-1C and exemplary heating portions of a surgical induction heating device, which as a group, have an overall shape similar to “T.” The heating portions depicted in FIG. 2 are located in different planes compared to the heating portions depicted in FIGS. 1A-1C. Comparing FIG. 2 and FIG. 1C, the two planes of heating portions intersect at different angles.

FIGS. 3A-3B depict different views of an exemplary surgical induction heating device with a disc-shaped heating portion and a tibial implant component. FIG. 3A illustrates a lateral view. FIG. 3B illustrates a superior view.

FIG. 4 depicts an oblique view of an exemplary surgical induction heating device with a disc-shaped heating portion and an exemplary tibial implant component. The heating portion is configured to heat a portion of the exposed surface of the implant.

FIGS. 5A-5D depict an exemplary surgical induction heating device, where the heating portions, as a group, have an overall shape similar to “C” and an exemplary knee femoral implant component. FIG. 5A illustrates an oblique view. FIG. 5B illustrates a lateral view. FIG. 5C illustrates a superior view. FIG. 5D illustrates a cross-sectional partial view. FIG. 5E depicts an exemplary knee femoral implant component.

FIG. 6 illustrates an oblique view of exemplary heating portions of a surgical induction heating device in close proximity to a knee femoral implant component. The heating portions are arranged concentric to one another.

FIG. 7 illustrates an oblique view of exemplary heating portions of a surgical induction heating device in close proximity to a knee femoral implant component. Two heating portions are arranged to intersect each other at a third heating portion.

FIG. 8 illustrates an oblique view of an exemplary curved square heating portion of a surgical induction heating device in close proximity to a knee femoral implant component.

FIG. 9 illustrates an oblique view of exemplary heating portions of a surgical induction heating device in close proximity to a knee femoral implant component. Four heating portions are substantially parallel to one another. The heating portions as a group form a shape similar to a fork.

FIG. 10 illustrates an oblique view of exemplary heating portions of a surgical induction heating device in close proximity to a knee femoral implant component. The implant contains two sets of heating portions, where the first set of heating portions are a mirror image of the second set of heating portions.

FIG. 11 illustrates a side view of an exemplary surgical induction heating device containing both C-shaped heating portions and disc-shaped heating portions, where the disc-shaped heating portions are in close proximity to a tibial implant component and the C-shaped heating portions are in close proximity to a knee femoral implant component simultaneously.

FIG. 12 depicts an exemplary surgical induction heating device containing a hemisphere-shaped heating portion in close proximity to an acetabular implant component.

FIG. 13A-13B depict an exemplary surgical induction heating device containing a cup-shaped heating portion in close proximity to a hip femoral stem implant component. FIG. 13A illustrates an anterior view. FIG. 13B illustrates a magnified partial superior view. FIG. 13C depicts an exemplary hip femoral stem implant component.

FIG. 14 illustrates a side view of an exemplary surgical induction heating device containing heating portions having a shape similar to an incandescent light bulb, in close proximity to an acetabular implant component and hip femoral stem component simultaneously.

### DETAILED DESCRIPTION OF THE INVENTION

### I. Systems

Systems described herein include an electrically conductive implant and a device for treating the implant via induction heating. The device contains one or more coils for providing an alternating magnetic field for induction heating. The device is a tool for performing specific actions, particularly for performing induction heating of an implant, typically during surgery or an operation.

### A. Induction Heating

The devices described herein can be used to provide a high frequency alternating magnetic field, which induces eddy currents in adjacent electrically conductive materials in an implant. The eddy currents can penetrate to a depth from the surface of the implant, and are thereby subjected to resistive losses based on the electrical properties of the implant material. The resistive losses can convert to heat. In this manner, the device provides precise amounts of localized heat in the implant, without causing collateral heating of the surrounding tissue.

The devices described herein can provide induction heating to an electrically conductive implant inside a patient's body. The coils are placed in close proximity to or contact with the implant and provide an alternating magnetic field to heat the implant, while allowing the implant to remain at its site during the treatment.

### B. Implants

### 1. Materials for Implants

The implant is electrically conductive. The implant contains one or more electrically conductive materials, such as metals or metal alloys.

### a. Materials

Examples of suitable conductive materials for the implants include, but are not limited to, silver, copper, gold, aluminum, tungsten, iron, platinum, tin, titanium, cobalt/chromium alloy, stainless steel, zirconium, and combinations thereof.

The implant generally does not contain a shape memory metal or metal alloy, such as nitinol and nitinol alloys.

### b. Properties

The amount of heat delivered to the implant is a function of the electrical properties of the implant material, such as resistivity and conductivity. The resistivity and conductivity of exemplary metals and metal alloys are provided in Table 1.

TABLE 1.
Conducting materials and their resistivity and conductivity at 20° C.

| | p (Ω · m) at | δ (S/m) |
|---|---|---|
| Material | 20° C. | at 20° C. |
| Silver | 1.59 × 10⁻⁸ | 6.30 × 10⁷ |
| Copper | 1.68 × 10⁻⁸ | 5.96 × 10⁷ |
| Gold | 2.44 × 10⁻⁸ | 4.52 × 10⁷ |
| Aluminum | 2.82 × 10⁻⁸ | 3.5 × 10⁷ |
| Tungsten | 5.60 × 10⁻⁸ | 1.79 × 10⁷ |
| Iron | 1.0 × 10⁻⁷ | 1.00 × 10⁷ |
| Platinum | 1.06 × 10⁻⁷ | 9.43 × 10⁶ |
| Tin | 1.09 × 10⁻⁷ | 9.17 × 10⁶ |
| Titanium | 4.20 × 10⁻⁷ | 2.38 × 10⁶ |
| Cobalt/Chromium alloy | 6.4 × 10⁻⁸ | 1.6 × 10⁷ |
| Stainless steel | 6.9 × 10⁻⁷ | 1.45 × 10⁶ |
| zirconium | 4.2 × 10⁻⁷ | 2.4 × 10⁶ |

### 2. Locations

The electrically conductive implants can have a suitable shape and size to be surgically inserted into a patient's body and implanted at a site in need thereof, such as the patient's knee, hip, shoulder, or elbow.

Implants at these locations are known in the art. The implant can be a knee prosthesis that is placed in and around the distal femur, and/or in the proximal tibia and on the tibial plateau. The implant can be a hip prosthesis that is placed within the acetabulum cup, and/or in and on the femur.

### 3. Components

The implant can be a unitary device or contain two or more components.

In some embodiments, the implant is a unitary device, i.e. contains a single component. Examples of implants that can be unitary devices include, but are not limited to, a knee femoral implant component, a tibial implant component, or an acetabular implant component. Optionally, the implant is a unicompartmental implant, which replaces only one side of a damaged joint. For example the implant can be a unicompartmental femoral component, for use in a partial knee replacement.

In some embodiments, the implant contains two or more components, optionally two components. Examples of implants that contain two or more components include, but are not limited to, an implant that contains a first component that is a knee femoral implant component and a second component that is a tibial implant component, and an implant that contains a first component that is a femoral stem implant component and a second component that is an acetabular implant component.

Optionally, the implant contains three components. For example, the implant can contain a first component that is an acetabular implant component, a second component that is a hip femoral stem implant component, and a third component that is a hip femoral ball implant component.

In an implant that contains more than one component, at least one of the components may be heated. In some embodiments, the method includes heating an exposed surface of each of the components in a multi-component implant. Optionally, one or more components in a multi-component implant is not heated. For example, in an implant containing three components, such as an acetabular implant component, a hip femoral stem implant component, and a hip femoral ball implant component, the acetabular implant component and hip femoral stem implant component are heated, but the hip femoral ball implant component is typically not heated.

### 4. Exposed Surfaces

The implant can be attached to a bone that is in a site in need of an implant, optionally the site is located adjacent to the knee, the hip, the shoulder, or the elbow of the patient.

The implant includes one or more bone contacting surfaces, and one or more exposed surfaces. The one or more bone contacting surfaces are attached to the bone at the site of implantation via any suitable fixation, such as via one or more fixation screws, placement of a bone cement, or placement of an osteogenic or osteoinductive material, or another biocompatible fixation material between the bone contacting surfaces and the surrounding bone surface.

The one or more exposed surfaces of the implant component are not in contact with or fixed to the surrounding bone at the location. In a multi-component implant, exposed surfaces of a first component can be in contact with exposed surfaces of another component of the implant. Optionally, one or more exposed surfaces in an implant or component of a multi-component implant are in contact with cartilage or a bone that is not the bone in which the implant component is fixed. For example in a total knee replacement, damaged bone and cartilage are removed and replaced with implant components that recreate the surface of the joint, i.e. the femoral component, and the tibial component. The exposed surfaces include surfaces that move as the knee bends and include surfaces that contact a spacer between the femoral component and tibial component.

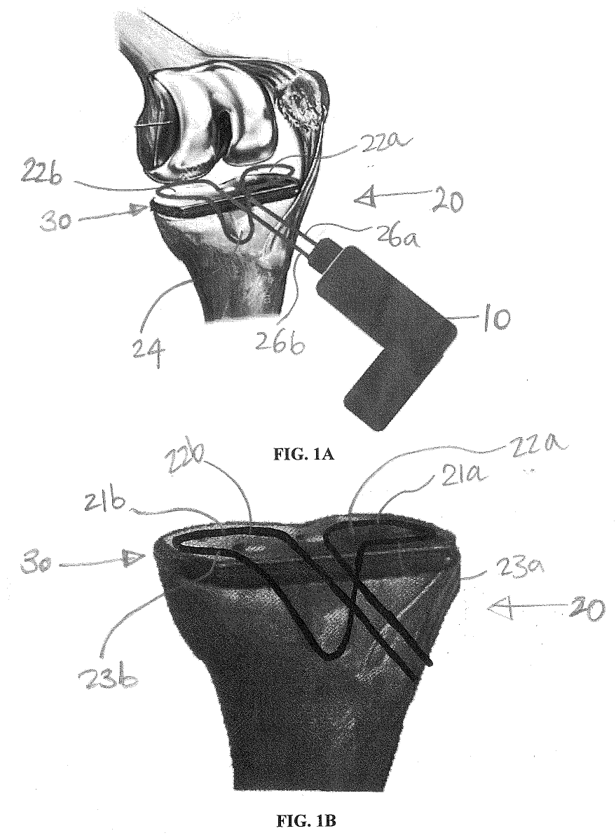

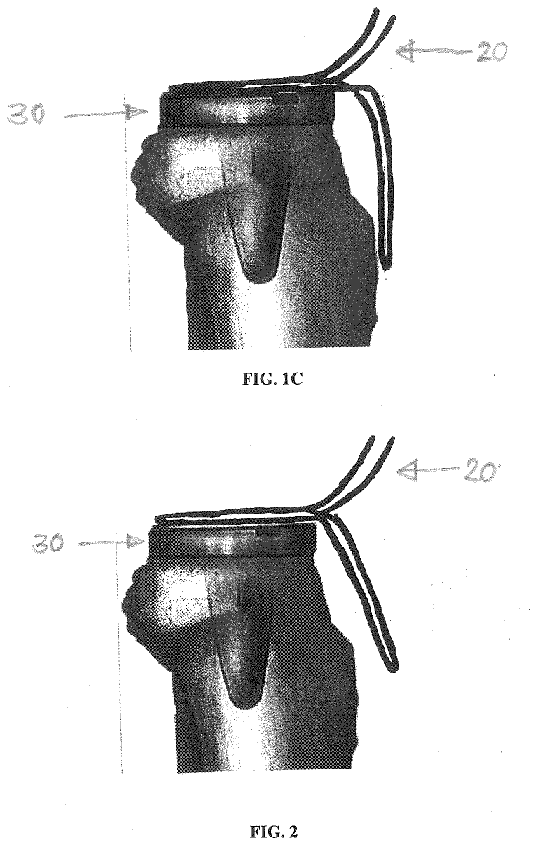

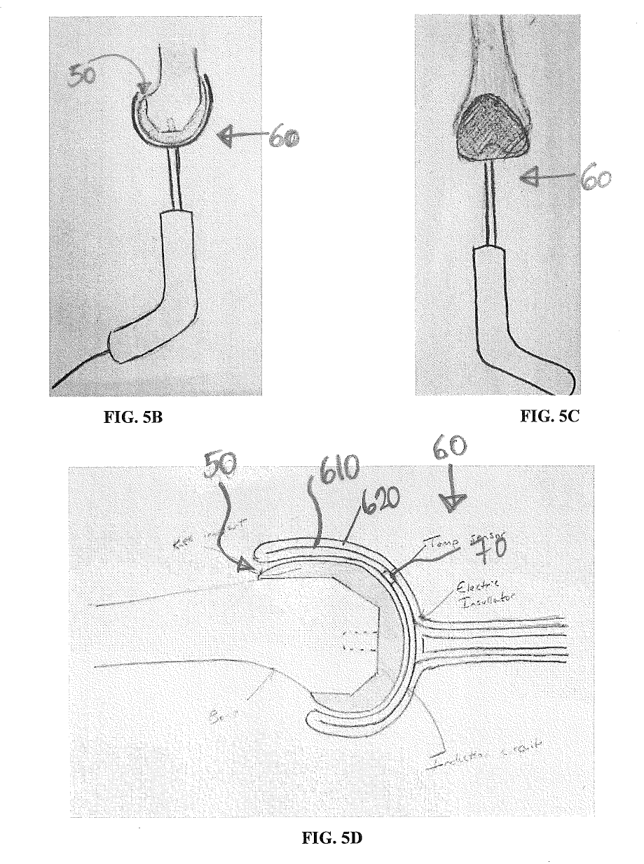

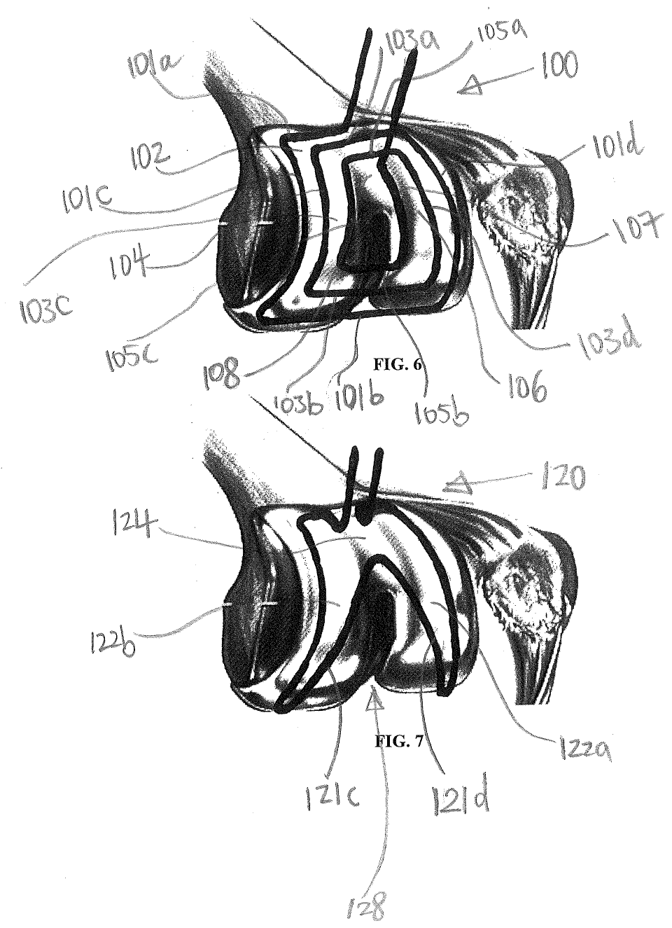

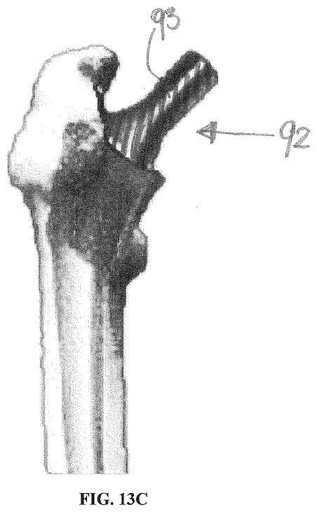

Exposed surfaces (marked with hatching) of exemplary implants are indicated in FIGS. 1D, 5E, and 13C. FIG. 1D depicts an exemplary tibial implant component 30 with exposed surface 31 (marked with hatching). FIG. 5E depicts an exemplary knee femoral implant component 50 with exposed surfaces 51a-51f (posterior femoral condyles 51a and 51b, distal femoral condyles 51c and 51d, and anterior flange 51e and 51f; marked with hatching). FIG. 13C depicts an exemplary hip femoral stem implant component 92 with exposed surface 93.

Typically the implant includes a stem portion, which is fixed in the surrounding bone at the site of implantation.

When cemented fixation is used, the implant components are commonly held in place with a fast-curing bone cement, such as polymethylmethacrylate (PMMA). When an implant is inserted into bone, both the bone and the cement must lock together in order to make the insertion last. Bone cement acts as a filler between the bone and the implant. Bone cement extrudes into the honeycomb structure of the internal bone.

When cementless fixation is used, each implant component can be “press-fit” onto the bone. This type of fixation relies on new bone growing into the surface of the implant. Cementless implants are typically made of or coated with a material that attracts new bone growth. Most are textured or coated so that the new bone grows into the surface of the implant. For example the stem of an implant can be a porous textured surface, optionally coated with an osteogenic or osteoinductive material.

Optionally hybrid fixation may be used, which combines cement fixation for one or more components in an implant and cementless fixation for other components in the implant. For example, in a total knee replacement, the femoral component can be inserted without cement, while the tibial and patellar components are inserted with cement.

Suitable bone cements include polymethylmethacrylate (PMMA) to affix the implant component, to the surrounding bone surface.

Optionally the implant components are textured or have a porous surface coating around much of the implant that allows new bone to grow into the surface of the implant. These implants can utilize cementless fixation, and utilize new bone growth for fixation to the surrounding bone.

Suitable osteogenic or osteoinductive materials can be placed in an opening or porous substrate of the implant to facilitate bone growth into the implant. Such materials include, but are not limited to, autograft, allograft, xenograft, demineralized bone, synthetic and natural bone graft substitutes, such as bioceramics and polymers, and osteoinductive factors. Optionally a carrier holds the materials within the cavities of the implants. These carriers can include collagen-based carriers, bioceramic materials, such as BIOGLASS®, hydroxyapatite and calcium phosphate compositions.

### C. Devices

The device contains one or more coils. The device has a suitable size and shape for the coils to be surgically inserted into a patient's body.

Optionally, the device can contain one or more temperature sensors, a power supply, and/or a hand unit.

### 1. Coils

The one or more coils can provide an alternating magnetic field. Each of the coils contains an electrically conductive material. The coils are generally sterile or can be sterilized prior to insertion. The coils may be disposed after a single use or multiple uses. After each use, the coils are typically sterilized.

Typically, all of the one or more coils are formed from the same unitary wire (i.e., a continuous wire), which includes two functionally different portions. The one or more coils include one or more connecting portions and one or more heating portions. The heating portions are the parts of the coil in close proximity to or contact with the exposed surfaces of the implant. “Close proximity” as used herein generally refers to a distance≤5 cm, such as within 5 cm, within 4 cm, within 3 cm, within 2 cm, within 1 cm, or within 0.5 cm of an exposed surface of the implant.

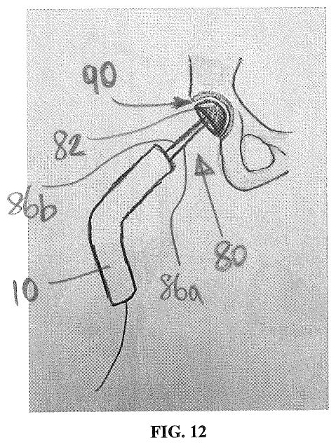

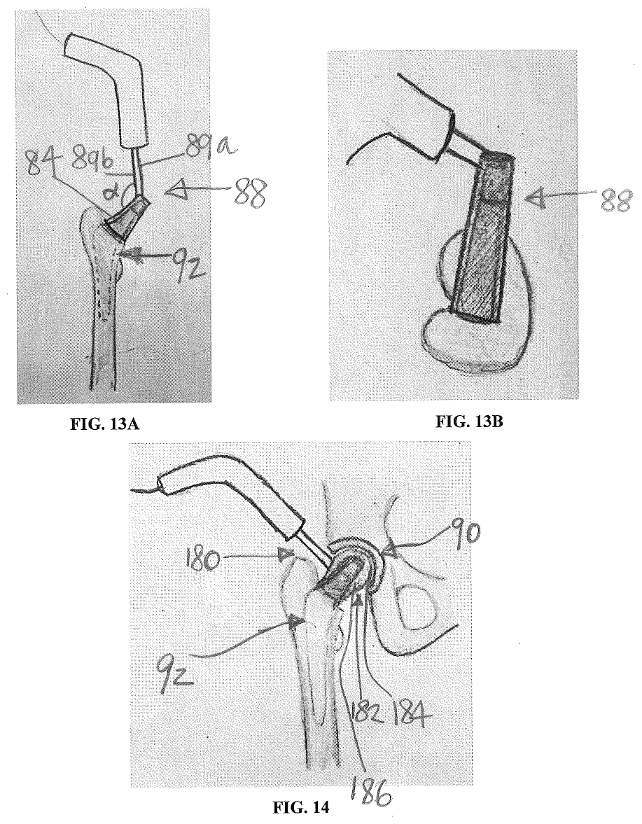

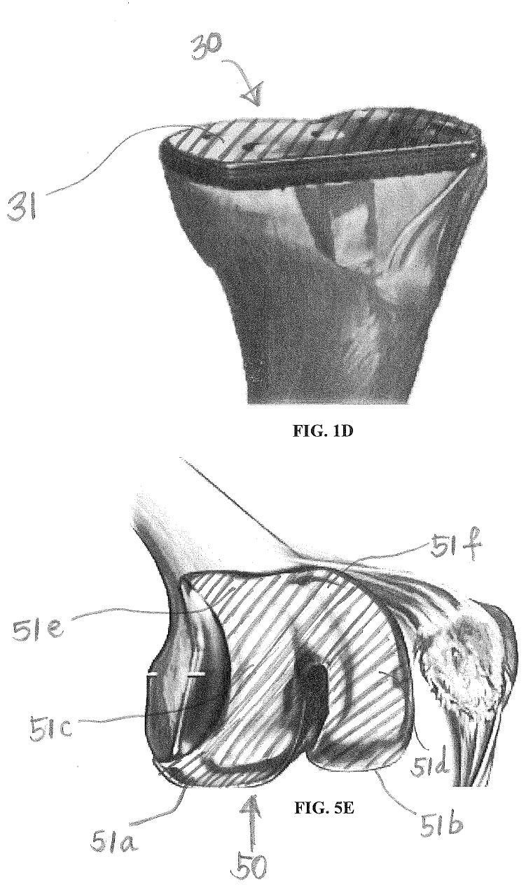

For example, the heating portions can be in contact with one or more exposed surfaces of an implant (e.g., see FIGS. 1C, 3A, and 4). The heating portions can be placed within about 5 cm, within about 4.5 cm, within about 4 cm, within about 3.5 cm, within about 3 cm, within about 2.5 cm, preferably within about 2 cm, within about 1.5 cm, within about 1 cm, or within about 0.5 cm of one or more exposed surfaces of the implant (e.g., see FIGS. 2A and 12).

Optionally, the heating portions can be placed within about 1 cm of one or more exposed surfaces of the implant, such as within about 90 mm, within about 80 mm, within about 70 mm, within about 60 mm, within about 50 mm, within about 40 mm, within about 30 mm, within about 20 mm, within about 10 mm, within about 5 mm, within about 2 mm, or within about 1 mm of one or more exposed surfaces of the implant.

### a. Materials for Coils

### i. Conductive Materials

The coils can be formed from any suitable conductive material. The conductive material may be biocompatible. If the material is not biocompatible, it is typically coated with a biocompatible coating. For example, the coil can be made from a metal or metal alloy conductor, such as copper, nickel, titanium, stainless steel, iron/chromium/aluminium alloy, nickel/chromium alloy, nickel/chromium/iron alloy, and a combination thereof.

The conductive material is typically in the form of a wire, such as a solid copper wire. The wire can have any suitable size and shape, as long as it is able to fit inside the patient's body and is insertable into the side in need of treatment. Suitable diameters are in the range of between about 0.1 mm and about 5 mm, between about 0.1 mm and about 5 mm, or between about 0.2 mm and about 1 mm.

### ii. Protective Coating

The one or more coils can contain a protective coating over the conductive material to prevent current outflow.

The protective coating is typically an electrical insulator. An electrical insulator is a material whose internal electric charges do not flow freely. Suitable electrical insulators include, but are not limited to, polyethylene, cross-linked polyethylene-XLPE, polyvinyl chloride-PVC, Teflon, and silicone.

The entire wire of the coil can be covered by the protective coating. Optionally, the heating portions of the coil are covered, while the connecting portion of the coil is uncovered.

The coil with a protective coating can have a diameter in the range of between about 1 mm and about 1 cm, between about 1 mm and about 0.5 cm, or between about 1 mm and about 0.1 cm.

### b. Connecting Portions

The connecting portions are closest to the user, and optionally attached to a handpiece. Moving away from the user, along the length of the coil, the connecting portions terminate in one or more heating portions. The connecting portion allows a user to manipulate the device to the desired location in the patient's body, to the site of the implant in need of treatment. The connecting portion typically extends from the handpiece for a suitable length and in a direction that allows the heating portions to be positioned in close proximity to or in contact with the exposed surfaces of the implant in the patient's body.

The one or more heating portions can follow the same direction as the connecting portions, i.e. remain in the same plane as the connecting portions. Alternatively, the coil can bend as it transitions from the connecting portion to the heating portions, such that the heating portions are located in one or more planes that are different from the plane of the connecting portion. For example, the heating portions or a portion thereof can be located in a plane that is at angle of less than 180°, for example, between 0° and 175° , between 0° and 170°, between 0° and 150°, between 0° and 120°, between 0° and 100°, or between 0° and 90° relative to the plane of the connecting portion.

### i. Length

The connecting portion can have any suitable length, as long as it is sufficient to position the heating portions in close proximity or contact with the exposed surfaces of the implant inside the patient's body. For example, the connecting portion can have a length at least about 5 cm, at least about 7 cm at least about 10 cm, at least about 15 cm, at least about 20 cm, at least about 25 cm, at least about 30 cm, at least about 35 cm, up to about 40 cm, up to about 35 cm, up to about 30 cm, up to about 25 cm, up to about 20 cm, up to about 15 cm, up to about 10 cm, between about 5 cm and about 40 cm, between about 10 cm and about 30 cm, or between about 15 and about 25 cm.

### ii. Shape

The connecting portion can be substantially straight or curved. Each of the connecting portions in a coil may be substantially straight or curved. Optionally, two or more of the connecting portions are substantially straight and one or more connecting portions are substantially curved. Alternatively, the connecting portions are all substantially straight or all substantially curved. Typically, the connecting portions are substantially straight.

Typically, the connecting portions are parallel or near parallel to one another.

### c. Heating Portions

The heating portions are the parts of the coil that are in close proximity or contact with the one or more exposed surfaces of the implant. Individually and together as a group, the heating portions have a suitable size and shape to fit into a patient's body.

Typically, as a group the heating portions have a shape that conforms to the shape of the one or more exposed surfaces of the implant. “Conform” generally refers to a shape that is complementary to the shape of the one or more exposed surfaces of the implant. For an implant with substantially flat exposed surfaces, one or more of the heating portions typically lie in substantially the same plane, forming a shape with a substantially flat surface. For an implant with a first substantially flat exposed surface and a second exposed surface that is located in a plane that is substantially perpendicular to the first exposed surface, a first portion of the one or more of the heating portions typically lie in substantially the same first plane, forming a shape with a substantially flat surface, and a second portion of the one or more of the heating portions can lie in substantially the same second plane, where the second plane is located at an angle of at least 90°, optionally greater than 90°, such as at least 100°, at least 110°, at least 120°, at least 130°, at least 145°, or at least 160°, optionally between 90° and 160°, such as between 90° and 145°, relative to the first plane.

### i. Shape

The coil can contain more than one heating portions. Optionally, the coil contains a single heating portion that is shaped to conform to the one or more exposed surfaces of the implant.

The shape of the heating portions are generally selected to maximize coverage of the exposed surfaces of the implant, thereby providing uniform heating of the implant without heating the surrounding tissues.

Each of the heating portions has a suitable shape and dimensions to fit inside the patient's body and be inserted into the site in need of treatment.

Adjacent heating portions typically include one or more spaces between each other. For example, as illustrated in FIG. 7, inner borders 121c and 121d define a space 128 between the heating portions 122a and 122b. As illustrated in FIG. 9, inner borders 141b and 141c define a space 149 between the heating portions 142 and 144.

Generally, the overall shape of the one or more heating portions as a group together with the one or more spaces conforms to the shape of one or more exposed surfaces of the implant.

Exemplary shapes for the heating portion include, but are not limited to, regular shapes, such as rectangle, square, circle, diamond, triangle, and irregular shapes. Each heating portion is generally defined by at least three sides of a shape.

Exemplary shapes of a group of heating portions include, but are not limited to, T-shaped, C-shaped, disc-shaped, hemisphere-shaped, cup-shaped, or a combination thereof. Optionally, the shape of a group of heating portions is not donut-shaped, e.g. is not in the general shape of a disc with a central hole.

In some embodiments, more than one heating portions are in a coil. Each of the heating portions may have the same, substantially the same, or different shapes. Optionally, two or more of the heating portions have a same or similar first shape and one or more heating portions have a second shape that is different than that of the first shape.

Optionally, the coil contains heating portions that have different shapes to conform to more than one component of an implant or more than one exposed surface in a single component of the implant. Optionally, one or more heating portions together have a first shape that conforms to a first component or first exposed surface of an implant, and one or more heating portions together have a second shape that conforms to a second component or second exposed surface of the implant.

Optionally, the coil contains one or more additional connecting portions arranged in any suitable position to connect heating portions for different components of an implant.

### ii. Number

The coil may include any number of heating portions (such as for example, from 1 to 1000) of any shape, to provide a sufficient magnetic field strength to the implant to generate a sufficient current flow to produce a desired temperature elevation in the exposed surface of the implant.

The number of heating portions in a coil is at least 1, and is optionally greater than 1, such as at least 2, at least 3, at least 4, at least 5, at least 6, at least 7, at least 8, at least 9, at least 10, at least 12, at least 15, at least 20, at least 22, at least 24, at least 25, at least 30, at least 32, at least 35, at least 40, at least 45, at least 50, at least 55, at least 60, at least 70, at least 80, at least 90, at least 100, at least 120, at least 140, at least 160, at least 180, at least 200, at least 300, at least 400, or at least 500. For example, a device contains up to 10 heating portions, such as 1, 2, 3, 4, 5, 6, 7, 8, 9, or 10 heating portions; optionally the device contains in the range of 10 to 100 heating portions, such as up to 20, up to 30, up to 40, up to 50, up to 60, up to 70, up to 80, up to 90, or up to 100; optionally the device contains in the range of 100 to 1000 heating portions, such as up to 150, up to 200, up to 300, up to up to 400, up to 500, up to 600, up to 700, up to 800, up to 900, or up to 1000 heating portions.

### iii. Relative Positions

In embodiments in which more than one heating portions are in a coil, the heating portions can be arranged in any suitable position relative to one another, such that the a shape of the heating portions as a group conforms to the one or more exposed surfaces of the implant and is insertable into the patient and configured for placement adjacent to or at the site of the implant.

The heating portions in a coil can be in the same plane or different planes. Two or more heating portions that are located in the same plane may be arranged concentric to one another or substantially parallel to one another. Two or more heating portions arranged in the same plane or aligned with the same arc of curvature can be referred to as a section of heating portions, particularly when the device contains heating portions two or more different planes or having different arcs of curvature.

Optionally, one or more of the heating portions are arranged in the same or substantially the same first plane, and one or more of the heating portions are arrange in a second plane that is different than the first plane. The first plane and the second plane may be substantially parallel to each other, substantially perpendicular to each other, or intersect at any suitable angle. Optionally, the heating portions in a coil can be in more than two different planes, where section of the one or more heating portions are in the same, or substantially the same first plane and another section of the heating portions are in a plane that is substantially perpendicular to the first plane or is at another angle, such as at least 90°, optionally greater than 90°, such as at least 100°, at least 110°, at least 120°, at least 130°, at least 145°, or at least 160°, optionally between 90° and 160°, such as between 90° and 145°, relative to the first plane. Yet another section of heating portions may be arranged in a third plane that is at a different angle than the second plane relative to the first plane. Additional sections of heating portions can be arranged at other angles or with different arcs of curvature relative to the first section of heating portions.

Optionally a first section of heating portions is arranged having a first arc of curvature and a second section of heating portions is arranged having a second arc of curvature that is different than the first. Additional sections of heating portions can be arranged at other different arcs of curvature relative to the first section of heating portions.

Optionally, one or more heating portions in the same plane or substantially the same plane can be arranged in different sets, where the heating portions in each set can be arranged in any suitable manner to conform to one or more exposed surfaces of the implant or implant component. Optionally, a mirror image (a second set) of a first set of heating portions is arranged on the opposite side of the first set of heating portions.

In embodiments in which heating portions are arranged in different planes, the heating portions in each plane may have the same, substantially the same, or different arrangements. Optionally, two or more of planes have a same or similar first arrangement and one or more planes have an arrangement that is different than that of the first arrangement. In some embodiments, the arrangement of the heating portions in a second plane is a mirror image of the arrangement of the heating portions in a first plane.

### iv. Location Relative to Implants

Generally, the heating portion(s) in a coil can be located at any suitable location relative to the implants. Optionally, the heating portion(s) are located superior to, inferior to, wrap around, and/or surround one or more exposed surfaces of at least one component of the implant.

For example, for a patient has a total or partial knee replacement, the heating portions(s) are placed in a position around the exposed surfaces of a knee femoral implant component, or superior to the exposed surfaces of a tibial implant component. Optionally, the heating portion(s) are located at a first position around and at a second position superior to the exposed surfaces of a knee implant containing both components.

For a patient with a total or partial hip replacement, the heating portion(s) are placed in a position within the cup of an acetabular implant component, or surround the exposed surfaces of a hip femoral implant component. Optionally, the heating portion(s) can locate in a first position within the cup and at a second position surrounding the exposed surfaces of a hip implant containing both components.

The heating portion(s) may physically contact one or more of the exposed surfaces of the implant. Optionally, the heating portion(s) are adjacent to one or more of the exposed surfaces of the implant and close to but do not physically contact any exposed surfaces of the implant. Optionally, the heating portion(s) are adjacent to one or more of the exposed surfaces of the implant and are in physical contact with one or more of the exposed surfaces of the implant.

### v. Coverage of Exposed Surfaces

The geometry of a heating portion can be defined by at least three sides of a shape. Typically, at least three sides of a heating portion define a boundary volume. In embodiments in which more than one heating portion are in a coil, the boundary volume is defined by at least three outer borders of the heating portions. The border may be straight or curved.

The boundary volume typically covers at least 70%, at least 75%, at least 80%, at least 85%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the exposed surface area of an implant.

Alternatively, the boundary volume covers a smaller portion of the exposed surface area, such as up to 50%, up to 45%, up to 40%, up to 35%, up to 30%, up to 25%, up to 20%, up to 15%, up to 10%, up to 5%, such as from 5% to 30%, from 5% to 25%, from 5% to 20%, from 5% to 15%, from 10% to 50%, from 10% to 30%, from 10% to 20% of the exposed surface area of an implant. Thus, the heating portion or heating portions is able to provide heating at a particular location of the exposed surfaces of an implant.

### d. Exemplary Coils of Exemplary Devices

Exemplary coils of exemplary devices are shown in FIGS. 1A-1C, 2, 3A-3B, 4, 5A-5D, 6-12, 13A-13C, and 14.

### i. Knee Prosthesis

### (1) Tibial Implant Component

FIGS. 1A-1C and 2 depict exemplary devices containing different shaped heating portions in a coil for heating a tibial implant component 30.

As shown in FIGS. 1A and 1B, the coil 20 contains three heating portions. The heating portions as a group have a “T” shape. The first and second heating portions 22a and 22b are in the form of a first loop and a second loop, where the first and second loops are located in the same plane (a first plane) and opposite each other. A third heating portion 24 is in the form of a third loop, where the third loop is located in a different plane (a second plane) than the plane of the first and second loops, which intersects with the first plane at any suitable angle, such as less than 180°, less than 150°, less than 120°, less than 90°, from 60° to 180°, from 60° to 150°, from 60° to 120°, or from 60° to 90°. Two connecting portions 26a and 26b are parallel to each other. The connecting portion 26a terminates with the first heating portion 22a, and the connecting portion 26b terminates with the second heating portion 22b. The connecting portions extend the first and second heating portions 22a and 22b longitudinally from a hand unit 10, such that the heating portions 22a, 22b, and 24 can be positioned in contact or in close proximity to the exposed surfaces of the tibial implant component 30. In use, and as shown in the figures, heating portions 22a and 22b are placed such that they are superior to and align in a plane that is parallel or substantially parallel with the exposed surfaces of the tibial implant component 30 (horizontal plane). Heating portion 24 is located in another plane at an angle of approximately 90° (FIG. 1C) or greater than 90° (FIG. 2) relative to the plane of the first and second heating portions 22a and 22b.

The configuration of the coil 20 permits the heating portions 22a and 22b to be positioned directly on top of the tibial implant component 30, with the third heating portion 24 at an angle of 90° or greater relative to the horizontal plane of the tibial implant component 30. The heating portions are in contact with and/or in close proximity to (but not in direct contact with) the exposed surface of the tibial implant component 30.

The outer borders 21a, 21b, 23a, and 23b of the first and second loops define boundary volumes of heating portions 22a and 22b, which together cover at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the surface area of the exposed surface of the tibial implant component 30.

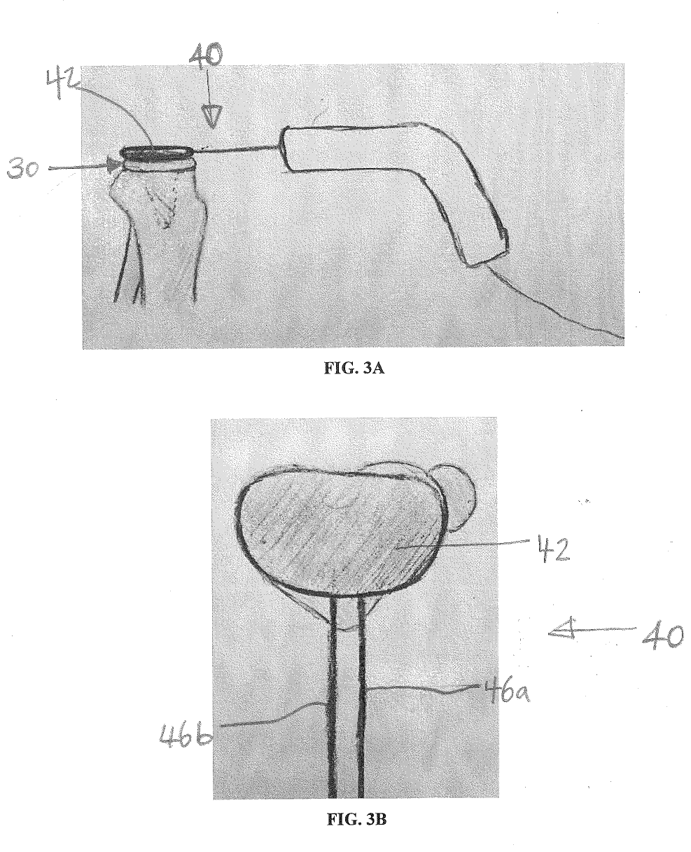

As shown in FIGS. 3A and 3B, the coil 40 contains a disc-shaped heating portion 42 in direct contact with the tibial implant component 30. Two connecting portions 46a and 46b are parallel to each other (see FIG. 3B). The connecting portions 46a and 46b terminate with the heating portion 42. The connecting portions extend the heating portion 42 longitudinally from hand unit 10, such that the heating portion 42 can be appropriately positioned in contact or in close proximity to the exposed surfaces of the tibial implant component 30. Heating portion 42 has a disc-shaped geometry and the geometry defines a boundary volume.

The boundary volume of the disc heating portion 42 (the shaded area in FIG. 3B) covers at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the exposed surface area of the tibial implant component 30.

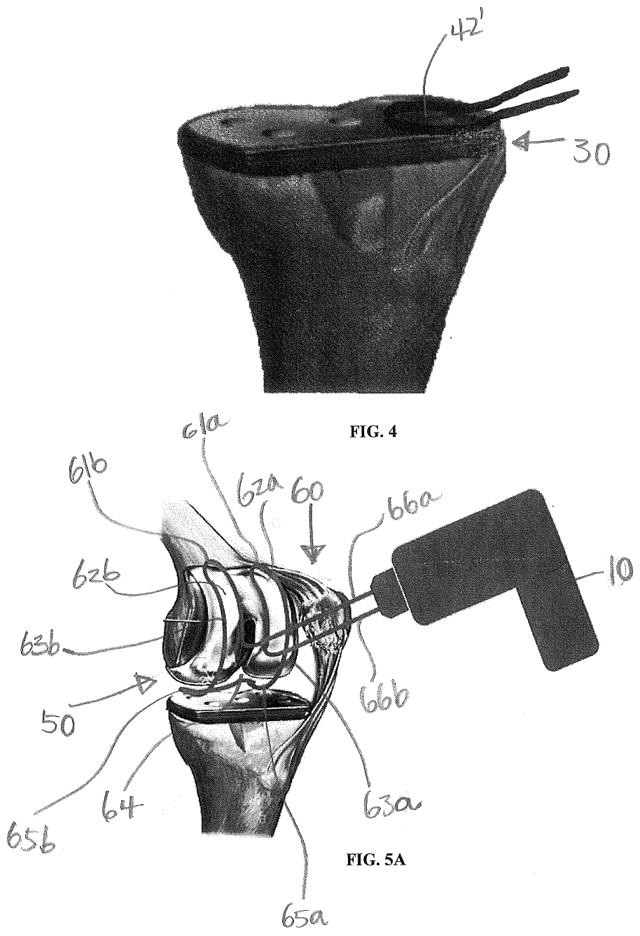

Alternatively, as shown in FIG. 4, the boundary volume of disc heating portion 42′ covers up to 50%, up to 45%, up to 40%, up to 35%, up to 30%, up to 25%, up to 20%, up to 15%, up to 10%, up to 5%, such as from 5% to 30%, from 5% to 25%, from 5% to 20%, from 5% to 15%, from 10% to 50%, from 10% to 30%, from 10% to 20% of the exposed surface area of the tibial implant component 30. Such a boundary volume permits the heating portion 42′ to provide heating at a specific location on the exposed surfaces of the tibial implant component 30.

### (2) Knee Femoral Implant Component

FIGS. 5A-5D and 6-10 depict exemplary devices containing heating portions in a coil for heating a knee femoral implant component 50.

As shown in FIG. 5A, the coil 60 contains three heating portions 62a, 62b, and 64. The heating portions as a group have a “C” shape. The coil contains two connecting portion 66a and 66b parallel to each other. The connecting portion 66a terminates with the first heating portion 62a, and the connecting portion 66b terminates with the second heating portion 62b. The connecting portions extend the first and second heating portions 62a and 62b longitudinally from a hand unit 10, such that the heating portions 62a, 62b, and 64 can be appropriately positioned in contact or in close proximity to the exposed surfaces of the knee femoral implant component 50. The first and second heating portions 62a and 62b are aligned substantially parallel to each other. The third heating portion 64 is substantially perpendicular to the heating portions 62a and 62b, and connects the first and second heating portions 62a and 62b. Heating portions 62a, 62b, and 64 together form the shape that wraps around the femoral implant component, such that the exposed surfaces including posterior femoral condyles 51a and 51b, distal femoral condyles 51c and 51d, and anterior flange 51e and 51f of the implant component are in contact with or close proximity to the heating portions. See also FIG. 5E.

The outer borders 61a, 61b, 63a, 63b, 65c, and 65d define a boundary volume of the heating portions 62a, 62b, and 64. The width from the outer border 63a to the outer border 63b is typically the same or substantially the same as the width of the knee femoral implant component 50, which generally has a width between about 5.5 and about 8.5 cm. Optionally, the width from outer border 63a to outer border 63b is at least 75%, at least 80%, at least 85%, or at least 90% of the width of the knee femoral implant component 50.

The boundary volume of the C-shaped heating portions 62a, 62b, and 64 (the shaded area in FIG. 5C) covers at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the exposed surface area of the knee femoral implant component 50.

FIG. 5D illustrates a magnified view of the lateral view of the C-shaped coil 60, covering the knee femoral implant component 50 placed around distal femur of a patient. The coil is made with a conductive wire 610 with a protective coating 620. A temperature sensor 70 is also included in the coil 60.

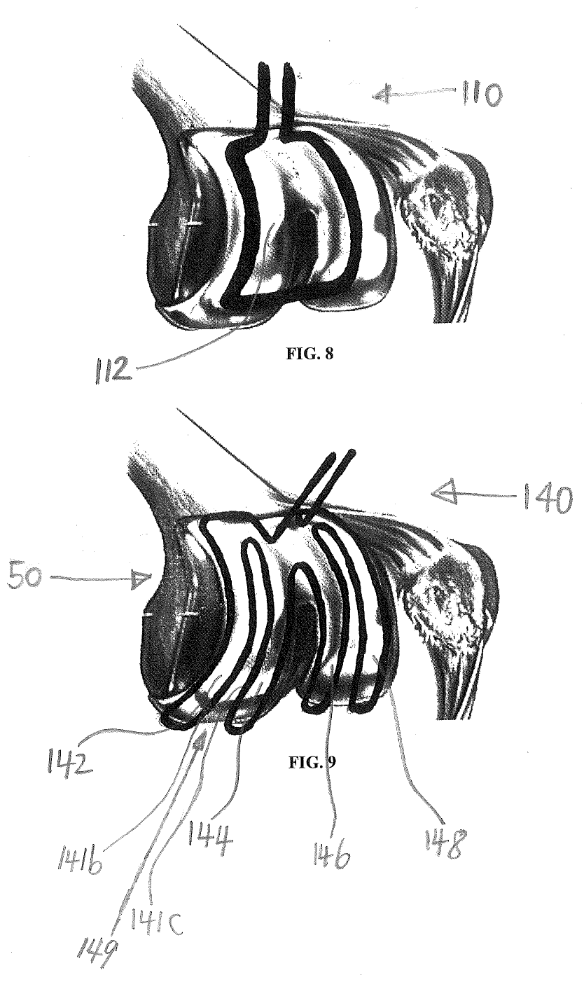

As shown in FIG. 6, the coil 100 contains four heating portions 102, 104, 106, and 108. The heating portions are substantially concentric to one another. Each heating portion is defined by at least three sides of a curved shape. For example, heating portion 102 is defined by outer borders 101a, 101b, 101c, and 101d; heating portion 104 is defined by inner borders 103a, 103b, 103c, and outer border 101d; heating portion 106 is defined by inner borders 105a, 105b, 105c, and 103d; heating portion 108 is defined by inner borders 105b, 105c, and 107. Optionally, the coil contains more than four heating portions that are substantially concentric to one another, such as at least 5, at least 10, at least 20, at least 50, at least 100 heating portions. In some embodiments, the coil 110 may contain only one heating portion 112 (e.g., see FIG. 8).

The width from the outer border 101c to the outer border 101d is typically the same or substantially the same as the width of the knee femoral implant component 50, which generally has a width in the range between about 5.5 and about 8.5 cm. Optionally, the width from outer border 101c to outer border 101d is at least 75%, at least 80%, at least 85%, or at least 90% of the width of the knee femoral implant component 50.

The outer borders 101a, 101b, 101c, and 101d define a boundary volume of the heating portions, which together cover at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the exposed surface area of the knee femoral implant component 50.

As shown in FIG. 7, the coil 120 may contain three heating portions 122a, 122b, and 124. The heating portions 122a and 122b intersect with each other at heating portion 124. The heating portions 122a and 122b may intersect with each other at any suitable angle, such that the inner border 121c and 121d intersect and has an angle less than 180°, less than 150°, less than 120°, less than 90°, from 5 to 180°, from 5 to 150°, from 5 to 12°, or from 5 to 90°, or from 5 to 60°.

As shown in FIG. 9, the coil 140 may contain four heating portions 142, 144, 146, and 148. The four heating portions are substantially parallel to one another. The heating portions together form a shape similar to a fork with four tines, which partially wrap around the knee femoral implant component 50.

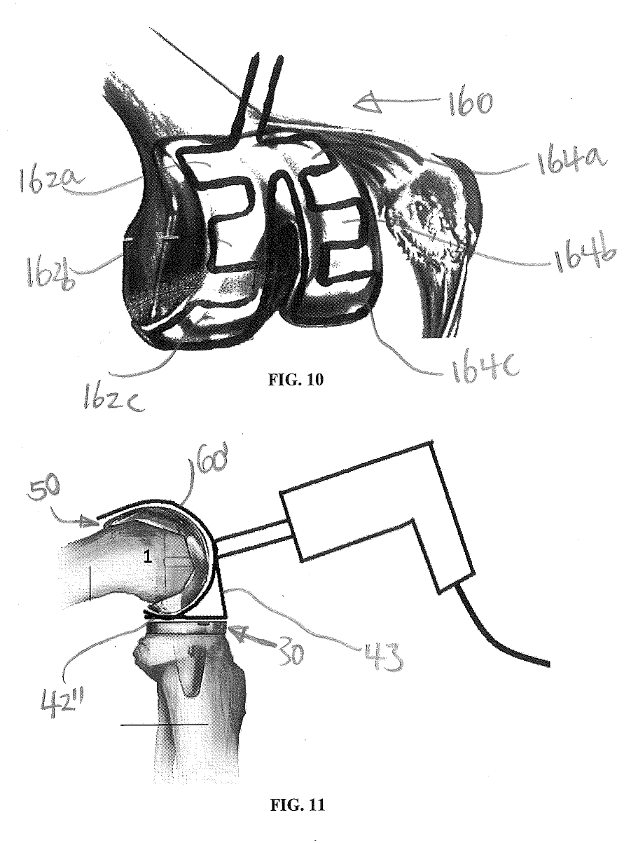

As shown in FIG. 10, the coil 160 may contain two sets of heating portions. The heating portions 162a, 162b, and 162c of a first set are parallel to one another. A mirror image (heating portions 164a, 164b, and 164c of a second set) of the first set of heating portions may be arranged on the opposite side of the first set.

Although not illustrated in FIGS. 5A-5D and 6-10, the coil may contain any number of heating portions of any shape, and may be arranged in any suitable positions such that taken together the heating portions as a group have a shape that conforms to the shape of the exposed surfaces of the knee femoral implant component.

### (3) Tibial and Knee Femoral Implant Components

The coil may contain heating portions that have different shapes to conform to more than one component of a knee implant.

For example, as shown in FIG. 11, the coil contains C-shaped heating portion(s) and disc-shaped heating portion(s). The disc-shaped heating portion(s) 42″ for the tibial implant component 30 can be incorporated into the C-shaped coil 60′ in any suitable position, such as into the posterior femoral condyles of the C-shaped coil 60′.

The coil may contain additional connecting portions to connect the heating portions for different components of an implant. For example, as shown in FIG. 11, the disc-shaped heating portion 42″ and the heating portions that form the “C” are connected by a third connecting portion 43, as depicted in FIG. 3.

### ii. Hip Prosthesis

### (1) Acetabular Implant Component

FIG. 12 depicts an exemplary device containing heating portion(s) in a coil for heating an acetabular implant component 90.

As shown in FIG. 12, the coil 80 contains a hemisphere-shaped heating portion 82. Two connecting portions 86a and 86b are parallel to each other, which terminate in the heating portion 82. The connecting portions extend the heating portion 82 longitudinally from a hand unit 10, such that the heating portion 82 can be appropriately positioned within the cup of the acetabular implant component 90. The heating portion 82 may be in contact or in close proximity to the exposed surfaces of the acetabular implant component 90.

The heating portion 82 is shaped as a hemisphere such that it can fit within the acetabular implant component 90 and conform to the exposed surface of the implant. The hemisphere-shaped heating portion 82 has a radius slightly smaller than the inner radium of the acetabular implant component 90. Optionally, the radius of the hemisphere-shaped heating portion 82 is smaller than the inner radius of the acetabular implant component 90 by about 1%, 2%, 3%, 4%, 5%, 6%, 7%, 8%, 9%, or 10%.

Typically, the outer surface area of the hemisphere-shaped heating portion 82, which faces toward the exposed surfaces of the implant, covers at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the surface area of the exposed surfaces of the acetabular implant component 90.

Although not depicted in FIG. 12, the coil 80 may contain any number of heating portions, such as more than one heating portion, arranged in any suitable positions such that taken together the heating portions as a group have a shape that conforms to the exposed surface of the acetabular implant component 90. Typically, the heating portions together as a group have a shape with a dimension that can fit within the cup of the acetabular implant component 90.

### (2) Hip Femoral Stem Implant Component

FIGS. 13A-13B depict an exemplary device containing heating portion(s) in a coil for heating a hip femoral stem implant component 92.

As shown in FIG. 13A, the coil 88 contains a cup-shaped heating portion 84. Two connecting portions 89a and 89b are substantially parallel to each other, and terminate in the heating portion 84. The connecting portions 89a and 89b extend the heating portion 84 from a hand unit 10, such that the heating portion 84 is positioned to substantially cover the exposed surfaces of the hip femoral stem implant component 92. The cup configuration of the heating portion 84 has sufficient dimensions and defines a sufficient volume inside the cup to surround the exposed surfaces of the hip femoral stem implant component 92. The heating portion 84 may be in contact with or in close proximity to the exposed surfaces of the hip femoral stem implant component 92. The heating portion 84 typically forms an angle (α) relative to the connecting portions 89a and 89b. This angle can any suitable angle to facilitate placement of the heating portion 84 to cover the exposed surfaces of the hip femoral stem implant component 92. In some instances, the angle can provide better visibility of the implant and ease of operation for the user during a surgical procedure.

The configuration of the heating portion 84 defines a boundary volume, which covers at least 70%, at least 75%, at least 80%, at least 90%, at least 95%, or approximately 99%, such as from 70% to 90%, from 70% to 80%, from 70% to 95%, or from 70% to 99%, from 80% to 90%, from 80% to 85%, from 80% to 95%, or from 80% to 99% of the exposed surface area of the hip femoral stem implant component 92.

Although not depicted in FIGS. 13A-13B, the coil 88 may contain any number of heating portions, such as more than one heating portion, arranged in any suitable positions such that taken together the heating portions as a group have a shape that conforms to the exposed surfaces of the hip femoral stem implant component 92.

### (3) Acetabular and Hip Femoral Stem Implant Components

The coil may contain heating portion(s) that have a shape to conform to more than one component of a hip implant.

For example, as shown in FIG. 14, the coil 180 contains a heating portion 182 having a shape similar to a light bulb. The round bottom 184 of the heating portion 182 can fit within the cup of the acetabular implant component 90 to cover the exposed surfaces of the implant 90, and the cup-shaped portion 186 of the heating portion 182 can surround the exposed surfaces of the hip femoral implant component 92.

Optionally, the coil may contain more than one heating portions and additional connecting portions to connect the heating portions for acetabular implant component 90 and the hip femoral implant component 92. For example, the coil may contain a first group of heating portions that have a shape to conform to the acetabular implant component 90, and a second group of heating portions that have a shape to conform to the hip femoral implant component 92, and one or more additional connecting portions to connect the first and second groups of heating portions in any suitable fashion.

### 2. Optional Components

### a. Temperature Sensors

The device can contain one or more temperature sensors for measuring the temperature of the one or more exposed surfaces of the implant.

The temperature sensors can be any type of sensors capable of detecting temperature (e.g., a thermometer, thermistor, and thermocouple, among others). Typically, the temperature sensors are one or more thermometers.

Typically, the temperature sensor(s) are incorporated in or attached to at least one of the one or more coils. The temperature sensor(s) are configured such that they can be in contact or in close proximity to the exposed surfaces of an implant. An exemplary temperature sensor positioned within the coil is depicted in FIG. 5D.

### b. Power Supply

The device may contain or is able to electrically connect with a power source to provide sufficient power to the device. Typically, the power supply is in electrical communication with the one or more coils.

The power supply may be a standard induction power supply, which converts outlet alternating current to a frequency suitable for the coil. Alternatively, the power supply is a capacitor, such as a disposable or rechargeable battery or battery pack. In some embodiments, the power supply is a combination of a standard induction power supply and a capacitor.

In embodiments where the power supply is a standard induction power supply, the power supply may include a male or female plug, which can be electrically coupled to a corresponding female or male plug that is attached or is able to attach to the device.

### i. Energy Characteristics

### (1) Power Output and Frequency

Typically, the power supply is between about 1 W and about 5 kW, and capable of operating at a frequency between about 50 kHz and about 1 MHz.

The power supply may provide continuous or pulsed energy to the coils.

In the continuous power output case, the power output may be constant or adjusted during use to maintain a desired temperature of the exposed surfaces of an implant.

In the pulsed power output case, the power output may be pulsed with a predetermined duration, frequency, and power level. For example, pulses can have a duration of 1 millisecond to 3 minutes with frequency of 0.1 Hz to 10 Hz. A pulsed energy activation can result in effective uniform heating of the implant, and limit the potential for inadvertent collateral damage to surrounding tissue.

### (2) Depth

The frequency of the power supply to generate eddy currents at a depth (δ, m) can be calculated as follows:

$$δ = \sqrt{\frac{ρ}{π \cdot μ \cdot f}} \qquad formula ( 1 )$$

where ρ is the resistivity of the implant (Ω·m), μ is the magnetic permeability

(H/m), and f is the frequency (Hz) of the power supply. Common conducting materials and their ρ and δ are presented in Table 1 above.

### c. Hand Unit

The device may contain a hand unit. The one or more coils are attachable to and removable from the hand unit.

Typically, the hand unit can connect the power supply and the one or more coils, allowing surgical manipulation.

The hand unit can have any suitable shape and size such that a user may hold it and appropriately position the coil to be in contact or in close proximity to one or more exposed surfaces of an implant.

### i. Materials for Hand Unit

The hand unit is typically made from a non-conductive material, such as plastic or non-conductive polymers. The hand unit can be sterilized for reuse or constructed of materials that permit it to be a disposable single-use component.

### ii. Gripping Structures

The hand unit can have gripping structures (e.g., knurls, ridges, rubber, etc.) to stabilize it in the hand of the surgeon. Optionally, the hand unit can contain a longitudinal ridge to register the rotational orientation of the hand unit within the hand of the surgeon.

### iii. Control

The hand unit can contain a control for activating/deactivating the power supply (i.e., to allow the power to be turned on and off), and/or adjusting the power output.

The control for adjusting the power output allows manual setting of the desired parameters for the power relative to the induction coil attached thereto, the shape and size of the implant, and the temperature feedback.

The control may have single-function, such as controlling the activation/deactivation of the power supply, or adjusting the power output. Alternatively, the control may have dual-function, such as controlling the activation or deactivation of the power supply, and adjusting the power output.

The control can be in any suitable form, such as a button or a switch. Alternatively, the device contains a foot switch that is separate from the hand unit but in electrical communication with the power supply and the hand unit to control the activation or deactivation of the power supply.

### iv. Cord

The hand unit may contain a cord, which can be electrically coupled to the power supply. For example, the cord includes a male or female plug at its proximal end, which can be electrically coupled to a female or male plug of a standard induction power supply.

Optionally, the cord is long enough (e.g., 2-10 meters) such that it allows the sterilized hand unit to be used in the operative field while a portion of the cord and the power supply are kept unsterile away from the operative field. “Operative field” as used herein refers to the field where the surgery is performed.

### v. Processor

Optionally, the hand unit can contain a processor, to automatically adjust the power and/or frequency of the power supply, in response to the signal provided from the temperature sensors. Alternatively, the processor is in another device that is separate from the hand unit, such as a smart phone or computer.

For example, the temperature sensors can provide the information in the form of a signal to a processor in the hand unit or in another device, such as a smart phone or computer. This way, the temperature sensors can provide direct temperature feedback in order to thermoregulate the induction power. If the temperature of one or more exposed surfaces of the implant measured by the temperature sensors exceeded a threshold, the processor in the hand unit can automatically reduce the power and/or frequency of the power supply, in order to reduce the eddy current, thus reduce the heating.

The processor may control the duration for heating.

For example, the processor can control the power supply to apply a predetermined power level continuously for a predetermined time period. Alternatively, the processor can control the power supply to apply one or more pulses of power to the implant for one or more of a predetermined time period, pulse duration, pulse repetition frequency, and at a predetermined power level.

### vi. Display

The hand unit may contain an output display. Alternatively, the display is in another device that is separate from the hand unit, such as a smart phone or computer.

Typically, the processor and the display of the hand unit or in another device, can be connected by wires or in wireless electrical communication, such as near infrared or blue tooth. The processor can receive signals from the temperature sensors and transmit it to the display, where the output display presents a number. Optionally, the display can present power output, frequency, time period of heating, and/or the temperature of the implant, as sensed by the sensor.

Optionally, an audible signal can be provided when the device is used for a pre-set time period or a desired temperature or temperature range is sensed by the sensor.

### II. Methods of Using the System

### A. Treating Electrically Conductive Implants

Methods of using the device for treating an electrically conductive implant in a patient's body are disclosed.

Generally, the method includes (i) surgically providing access to one or more exposed surfaces of an implant, (ii) placing the one or more heating portions of the one or more coils in a first position, where the first position is in close proximity to or in contact with the one or more exposed surfaces of the implant, and (iii) activating the power supply to provide an alternating magnetic field to heat the implant.

### 1. Surgically Providing Access to the Exposed Surfaces of the Implant

Generally, prior to inserting one or more heating portions of the device into the patient's body, surgical access to the one or more exposed surfaces of the implant may be provided. Any suitable surgical procedure can be used to provide access for inserting the one or more heating portions to the desired site.

Exemplary surgical procedures to provide access for inserting the one or more heating portions include, but are not limited to, anterior approach to the hip, anterolateral approach to the hip, lateral approach to the hip, posterior approach to the hip, medial parapatellar approach to the knee, subvastus approach to the knee, mid vastus approach to the knee, and arthroscopic approach to the knee.

### 2. Placing the Heating Portions

Generally, a user places one or more heating portions in a first position inside the patient's body through the surgically provided access. The first position is typically in close proximity to or in contact with one or more exposed surfaces of an implant.

The user can be a human, such as a medical professional, or a robot. The placement may be directly directed by the human. The placement may be through a robotic arm directed by a computer that is programmed or by a human who operates the robotic arm.

### a. Distance

Typically, the heating portions are placed at a distance of about 5 cm or less from one or more exposed surfaces of an implant. For example, the one or more heating portions are placed in contact with the one or more exposed surfaces of the implant (e.g., see FIGS. 1C, 3A, and 4)

Alternatively, the heating portions are not in contact with the one or more exposed surfaces of the implant, but are placed within about 5 cm, within about 4.5 cm, within about 4 cm, within about 3.5 cm, within about 3 cm, within about 2.5 cm, preferably within about 2 cm, within about 1.5 cm, within about 1 cm, or within about 0.5 cm of one or more exposed surface of the implant (e.g., see FIGS. 5A and 12).

Optionally, the heating portions can be placed within about 1 cm of one or more exposed surfaces of the implant, such as about 90 mm, about 80 mm, about 70 mm, about 60 mm, about 50 mm, about 40 mm, about 30 mm, about 20 mm, about 10 mm, about 5 mm, about 2 mm, or about 1 mm.

### b. Location

The one or more heating portions can be placed in close proximity to or in contact with one or more exposed surfaces of an implant in the knee, the hip, the shoulder, or the elbow of a patient's body.

For example, as shown in FIGS. 1A and 3A, one or more heating portions can be placed in close proximity to the exposed surfaces of a tibial implant component. As shown in FIG. 5A, more than one heating portion can be placed in close proximity to the exposed surfaces of a knee femoral implant component. As shown in FIG. 12, one or more heating portions can be placed in close proximity to the exposed surfaces within the cup of an acetabular implant component. As shown in FIG. 13A, one or more heating portions can be placed in close proximity to the exposed surfaces of a hip femoral stem implant component.

### 3. Activating the Power Supply

Generally, a user can activate the power supply. The user can be a human, who manually activates the power supply. Alternatively, the user can be a computer or robot, which is programmed to activate the power supply.

### a. Alternating Magnetic Field

Upon activation of the power supply, one or more coils provide an alternating magnetic field to one or more exposed surfaces of the implant.

The alternating magnetic field induces eddy currents in the implant, which cause heat and elevate the temperature of the one or more exposed surfaces of the implant.

The step of activating power supply and the step of placing one or more heating portions in a first position that is in close proximity to or in contact with one or more exposed surfaces of the implant may occur simultaneously, substantially simultaneously, or sequentially. Optionally, the activation of power supply may occur before or after placing one or more heating portions in the first position.

The power supply typically provides a power output between about 1 W and about 5 kW, and a frequency between about 50 kHz and about 1 MHz.

### 4. Optional Steps

### a. Maintaining the Heating Portions in Position for Heating the Implant

The method may include a step (iv) of maintaining the one or more heating portions in the first position for a sufficient period of time to achieve a predetermined temperature of the one or more exposed surfaces of the implant, and maintain the temperature for up to about 15 minutes.

A user, such as a human, can manually maintain the position of the one or more heating portions. Alternatively, the device can be clamped or otherwise mechanically stabilized to maintain the first position of the one or more heating portions. Optionally, the user is a robot, which can maintain the position of the heating portions with a robotic arm.

### i. Predetermined Temperature

Generally, the predetermined temperature of the one or more exposed surfaces of the implant is up to 160° C., up to 150° C., up to 120° C., up to 100° C., up to 85° C., up to 65° C., at least 40° C., at least 45° C., at least 50° C., at least 55° C., at least 60° C., at least 100° C., such as from 40° C. to 160° C., from 40° C. to 85° C., or from 100 ° C. to 160 ° C.

### ii. Period of Time

The heating portions are maintained in the first position for a sufficient period of time to both (1) achieve the predetermined temperature of the one or more exposed surfaces of the implant and (2) maintain the temperature.

The time to achieve a predetermined temperature of the one or more exposed surfaces may be short, such as up to 1 minute, up to 30 seconds, up to 15 seconds, up to 10 seconds, up to 5 seconds, or up to 1 second. Optionally, the time to achieve the predetermined temperature is long, such as at least 2 minutes, at least 2 minutes, at least 5 minutes, or at least 10 minutes.

After achieving the predetermined temperature of the one or more exposed surfaces of the implant, this temperature can be maintained for up to 10 minutes, up to 5 minutes, up to 4 minutes, up to 3 minutes, up to 2 minutes, up to 1 minute, or up to 30 seconds.

Optionally, the temperature is maintained for up to 15 minutes. A robotic arm can then be used to maintain the position of the heating portions of the device. Alternatively, the device may be clamped at a location by the user, such as a medical professional.

### b. Selecting One or More Coils from an Array of Coils

Optionally, a user selects one or more coils having heating portions with the desired shape for heating the implant at the site in need of treatment from an array of two or more coils having heating portions of different shapes, prior to step (i) of surgically providing access to the one or more exposed surfaces of an implant, or step (ii) of placing one or more heating portions of the device in a first position inside the patient's body.

### c. Attaching Coils to Hand Unit

Optionally the coils are attachable and removable from the hand unit. Thus, the same had unit can be used for a variety of different procedures to treat different implants or implant components at different sites in the same or different patients. The device may be prepared for insertion by first selecting one or more coils having heating portions with the desired shape for heating the implant at the site in need of treatment, and attaching the one or more coils to the hand unit prior to inserting the heating portions into the patient's body.

Attaching the one or more coils to the hand unit can facilitate surgical manipulation of the device by a user. For example, a user may hold the hand unit to insert, and manipulate the hand unit to place the one or more heating portions in close proximity to or in contact with one or more exposed surfaces of an implant.

Optionally, attaching the one or more coils to the hand unit allows a user to activate/deactivate, and/or adjusting the power output using a control in the hand unit.

### d. Selecting a Power Output

The method can include a step of selecting a power output. A user can select a power output suitable for heating a particular implant and/or for a particular application.

The user can select a power output simultaneously or substantially simultaneously while activating the power supply. Alternatively, the user can select the power output prior to inserting the one or more heating portions in a patient's body or activating the power supply.

The steps of selecting the power output and/or adjusting the power output can be automated in response to data from the one or more sensors to a control unit.

### e. Adjusting Power Output

The method can include a step of adjusting the power output of the device during step (iv) of maintaining the one or more heating portions in the first position for a sufficient period of time to achieve a predetermined temperature of the one or more exposed surfaces of the implant, and maintain the temperature for up to about 15 minutes. Optionally, a user can adjust the power output during heating to maintain the predetermined temperature of the one or more exposed surfaces of the implant.

A user, such as a human, may manually adjust the power output. Alternatively, the power output can be adjusted by a programmed processor in response to data from the one or more sensors to maintain a predetermined temperature or to modify the temperature of the surface of the implant.

### f. Moving Heating Portions to a Second Position

The method can include a step of moving the one or more heating portions to a second position after step (iv) described above.

The second position is different from the first position and is in close proximity to or in contact with one or more exposed surfaces of the implant.

A user typically maintains the one or more heating portions in the second position for a sufficient period of time to achieve a predetermined temperature of the one or more exposed surfaces of the implant, and maintain the temperature for up to about 15 minutes. The predetermined temperature at the second position may be the same as the predetermined temperature in the first position. Optionally, different treatments are needed at different locations of the exposed surfaces of the implant and thus, the method includes heating the exposed surfaces at the second position to a different temperature and/or for a different period of time than the predetermined temperature and period of time at the first position. This allows only one area at a time on the implant to be heated at a time, such as when different types of treatments or different treatment time periods are needed.

### B. Removing Biofilm and/or Reducing Bacterial Burden

The device may be used in a method for thermal removal of biofilm and/or reducing bacterial burden.

The method can include the step of surgically providing access to one or more exposed surfaces of an implant. This permits thermoregulation and surgical treatment of the joint space, thereby minimizes the chance for biofilm recurrence.

The implant or implant component to be treated typically does not move and remains at its implanted location throughout the method and following completion of the treatment. This avoids or decreases complications otherwise associated with moving the implant or removing the implant from the body for treatment, such as bone loss, loss of fixation, and/or long operative times.

In some embodiments, prior to placing the heating portions in the patient's body at or in close proximity to the surface to be headed, a non-conductive spacer or liner associated with an implant or one or more components in a multi-component implant is removed. Exemplary non-conductive spacer or liner include polyethylene spacers, such as a polyethylene spacer located in between a knee femoral implant component and a tibial implant component, or a plastic liner, such as a plastic liner covering a hip femoral ball implant component. For example, in a multi-component implant including a knee femoral implant component and a tibial implant component, a polyethylene spacer located in between the knee femoral implant component and the tibial implant component can be removed from the patient, prior to placing one or more heating portions in close proximity to or in contact with one or more exposed surfaces of the implant component(s) to be heated.

### 1. Method Steps

Typically, the method of removing biofilm and/or reducing bacterial burden follows the method steps described above.

For example, the user (i) surgically provides access to one or more exposed surfaces of the implant, (ii) places the one or more heating portions in a first position that is in close proximity to or in contact with the one or more exposed surfaces of an implant, and (iii) activates the power supply to provide an alternating magnetic field to heat the implant.

Optionally, the method can include any or all of the optional steps described above.

The user can (iv) maintain the one or more heating portions in the first position for a sufficient period of time to achieve a predetermined temperature of the one or more exposed surfaces of the implant, and maintain the temperature for up to about 15 minutes.

Optionally, the user can move the one or more heating portions to a second position after step (iv), where the second position is different from the first position, and where the second position is in close proximity to or in contact with the one or more exposed surfaces of the implant.

Optionally, in a multi-component implant, where one or more components are not heated, a user can remove one or more components that are not heated prior to placing one or more heating portions in close proximity to or in contact with one or more exposed surfaces of the implant. Optionally, the one or more components that are not heated can be replaced with one or more new components. For example, in a multi-component hip implant, such as one containing an acetabular implant component, a hip femoral stem implant component, and a hip femoral ball implant component, the hip femoral ball implant component can be removed from the patient, and the acetabular implant component and hip femoral stem implant component are heated. Optionally the hip femoral ball implant component is replaced with a new hip femoral ball implant component after heating of the hip femoral stem implant component and the acetabular implant component.

### a. Temperature

With respect to step (iv), the predetermined temperature is typically sufficiently high to remove biofilm from an implant and/or reduce bacterial burden on the implant.

Generally, the predetermined temperature of the one or more exposed surfaces of the implant is at least 40° C., at least 45° C., at least 50° C., at least 55° C., up to 100° C., up to 95° C., up to 90° C., or up to 85° C., such as between about 40° C. and about 100° C., between about 40° C. and about 95° C., or between about 40° C. and about 85° C.

### b. Period of Time

With respect to step (iv), the one or more heating portions are typically maintained in the first position and/or the second position for a period of time between about 30 seconds and about 10 minutes, such as between about 30 seconds and about 5 minutes.

The period of time is the total time sufficient to achieve the predetermined temperature of the one or more exposed surfaces of the implant, and to maintain the temperature to remove biofilm from an implant and/or reduce bacterial burden on the implant.

### c. Administering Antibiotic and/or Antimicrobial Agents

One or more antibiotic and/or antimicrobial agents may be administered to the patient throughout the method or at different intervals during the method. Optionally, an antibiotic and/or antimicrobial agent is administered prior to, during, and after any one or more of the steps described above.

Optionally, the method includes administering one or more antibiotic and/or antimicrobial agents to the patient prior to step (i), prior to or during step (iii), and/or during or subsequent to step (iv) described above For example, one or more antibiotic and/or antimicrobial agents may be administered to the patient prior to inserting one or more heating portions into the patient's body.

Alternatively, one or more antibiotic and/or antimicrobial agents may be administered to the patient prior to, or simultaneously with, or substantially simultaneously with, activating the power supply.

One or more antibiotic and/or antimicrobial agents may be administered to the patient simultaneously with, or substantially simultaneously with, or subsequent to heating the implant, such as during or after the step of maintaining the heating portions in the first and/or second position.

One or more antibiotic and/or antimicrobial agents may be administered to the patient throughout the method or at different intervals during the method. Optionally, an antibiotic and/or antimicrobial agent is administered prior to, during, and after any one or more of the steps described above.

### i. Administration Route

The one or more antibiotic and/or antimicrobial agents can be administered to the patient systemically, regionally, and/or locally.

One or more antibiotic and/or antimicrobial agents can be orally administered to the patient. For example, the one or more antibiotic and/or antimicrobial agents can be administered as liquids, powders or granules, suspensions or solutions in water or non-aqueous media.

Alternatively, one or more antibiotic and/or antimicrobial agents can be administered to the patient by intravenous injection or intraperitoneal injection.

Optionally, one or more antibiotic and/or antimicrobial agents are administered to the patient by locally applying the one or more antibiotic and/or antimicrobial agents on one or more of the exposed surfaces of the implant and/or the surrounding soft tissue where the implant is located in the patient's body.

### ii. Administration Amount

The amount of the one or more antibiotic and/or antimicrobial agents required will vary from subject to subject according to their need.

Typically, when used in combination with induction heating for treating biofilm on implants and/or reducing bacterial burden, the impact of heat on the biofilm and/or the bacteria augments the effect of a given dose of antibiotic and/or antimicrobial, optionally a lower the dose is required to treat an infected region or tissue compared to the dose required when administered alone, e.g. in the absence of the induction heating method.

### C. Disrupting Implant-Bone, Bone-Cement, and/or Implant-Cement Interface

Optionally, the device is used to heat an implant to facilitate implant removal during a revision surgery. For example, if the implant is fixed via an implant-cement or implant-bone interface before heating, following heating, the implant-bone, bone-cement, and/or implant-cement interface may be disrupted.

### 1. Method Steps

Typically, the method of disrupting implant-bone, bone-cement, and/or implant-cement interface follows the method steps described above.

For example, the user (i) surgically provides access to one or more exposed surfaces of the implant, (ii) places the one or more heating portions in a first position that is in close proximity to or in contact with the one or more exposed surfaces of an implant, and (iii) activates the power supply to provide an alternating magnetic field to heat the implant.

Optionally, the method can include any or all of the optional steps described above.

For example, the user can (iv) maintain the one or more heating portions in the first position for a sufficient period of time to achieve a predetermined temperature of the one or more exposed surfaces of the implant, and maintain the temperature for up to about 5 minutes.

Optionally, the user can move the one or more heating portions to a second position after step (iv), where the second position is different from the first position, and where the second position is in close proximity to or in contact with the one or more exposed surfaces of the implant.

### a. Temperature

With respect to step (iv), the predetermined temperature is typically sufficiently high to disrupt implant-bone, bone-cement, and/or implant-cement interface.

Optionally, the temperature is sufficiently high to result in loosen the implant from the surrounding bone due to compromise of the cement hardness/integrity, thereby disrupting the implant-cement and/or cement-bone interfaces.

Alternatively, the temperature is sufficiently high to disrupt the implant-bone interface, and thereby loosen the implant from the surrounding bone.

Generally, the predetermined temperature of the one or more exposed surfaces of the implant is at least 100° C., at least 110° C., at least 120° C., at least 130° C., at least 140° C., up to 160° C., up to 155° C., up to 150° C., or up to 145° C., such as between about 100° C. and about 160° C.

### b. Period of Time

With respect to step (iv), the one or more heating portions are typically maintained in the first position and/or the second position for a period of time between about 5 seconds and about 5 minutes.

The period of time is the total time sufficient to achieve the predetermined temperature of the one or more exposed surfaces of the implant, and to maintain the temperature to disrupt implant-bone, bone-cement, and/or implant-cement interface.

### c. Removing the Implant from Patient

Optionally, the method can include a step of removing the implant from the patient's body. Typically, implant is removed after step (iv).

The implant can be removed using any suitable removal tools, such as a slap hammer and a mallet. Optionally, a user removes the implant following loosening of the implant from the surrounding bone, optionally a user guides a robot or a robot is programmed to remove the implant from the patient's body.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A surgical device for heating an implant located in a patient's knee, hip, shoulder, or elbow via induction heating, wherein the device comprises
one or more coils,
wherein each of the one or more coils comprises one or more electrically conductive materials,
wherein the one or more coils comprise one or more connecting portions and one or more heating portions, and
wherein the one or more heating portions together as a group have a heating portion shape that conforms to one or more surfaces of the implant.

2. The device of claim 1, wherein the heating portion shape conforms to one or more surfaces of a knee femoral implant component, a tibial implant component, a hip femoral stem implant component, or an acetabular implant component.

3. The device of claim 1, wherein a first section of the heating portion shape conforms to one or more surfaces of a knee femoral implant component, and a second section of the heating portion shape conforms to one or more surfaces of a tibial implant component.

4. The device of claim 1, wherein
a first section of the heating portion shape conforms to one or more surfaces of a hip femoral stem implant component, and
a second section of the heating portion shape conforms to one or more surfaces of an acetabular implant component.

5. The device of claim 1, wherein the one or more heating portions are T-shaped, C-shaped, disc-shaped, hemisphere-shaped, cup-shaped, or a combination thereof.

6. The device of claim 1, further comprising one or more temperature sensors incorporated in or attached to at least one of the one or more coils.

7. The device of claim 1, further comprises a hand unit, and wherein the one or more coils are attachable to and removable from the hand unit.

8. A method of treating an electrically conductive implant in a patient's body using a device,
wherein the device comprises
one or more coils,
wherein each of the one or more coils comprises an electrically conductive material,
wherein the one or more coils comprise one or more connecting portions and one or more heating portions, and
wherein the one or more coils are in electrical communication with a power supply,
the method comprising
(i) surgically providing access to one or more exposed surfaces of the implant,
(ii) placing the heating portions of the one or more coils in a position,
wherein the position is in close proximity to or in contact with the one or more exposed surfaces of the implant, and
(iii) activating the power supply to provide an alternating magnetic field to heat the implant.

9. The method of claim 8, further comprising
(iv) maintaining the heating portions in the position for a sufficient period of time to achieve a predetermined temperature of the one or more exposed surfaces of the implant and maintain the temperature for up to about 15 minutes.

10. The method of claim 8, further comprising:
attaching the one or more coils to a hand unit prior to step (i).

11. The method of claim 8, wherein the implant is located in the knee, the hip, the shoulder, or the elbow of the patient's body.

12. The method of claim 8, wherein the one or more heating portions cover at least 75% of the surface area of the one or more exposed surfaces of the implant.

13. The method of claim 9, wherein during step (iv), the predetermined temperature is sufficiently high to remove biofilm from the implant and/or reduce bacterial burden on the implant.

14. The method of claim 9, wherein during step (iv), the predetermined temperature is sufficiently high to disrupt the implant-bone, bone-cement, and/or implant-cement interface.

15. The method of claim 9, wherein during step (iv), the predetermined temperature is between about 40° C. and about 85° C. or between about 100° C. and about 160° C.

16. The method of claim 9, wherein during step (iv), the heating portions are maintained in the position for a period of time between about 30 seconds and about 15 minutes.

17. The method of claim 8, wherein the implant is a knee femoral implant component, a tibial implant component, a hip femoral stem implant component, and/or an acetabular implant component.

18. The method of claim 8, wherein the implant contains more than one component,
wherein the first component is an acetabular implant component, and
wherein the second component is a hip femoral stem implant component.

19. The method of claim 8, wherein the implant contains more than one component,
wherein the first component is a knee femoral implant component, and
wherein the second component is a tibial implant component.

20. A system comprising
an electrically conductive implant, and
a device for treating the implant via induction heating,
wherein the device comprises
one or more coils,
wherein each of the one or more coils comprises one or more electrically conductive materials.
